(19) United States
(12) Patent Application Publication
(10) Pub. No.: US 2020/0261422 A1
(43) Pub. Date: Aug. 20, 2020

(54) METHODS FOR EVALUATING TREATMENTS AND PHYSIOLOGY IN HUMAN PATIENTS USING INTRAVENOUS ALPHA-2 ADRENERGIC ANTAGONIST AGENTS

(71) Applicant: Massachusetts Institute of Technology, Cambridge, MA (US)

(72) Inventors: Jacob A. Donoghue, Boston, MA (US); Emery N. Brown, Brookline, MA (US); Earl K. Miller, Somerville, MA (US); Noah D. Donoghue, Nantucket, MA (US)

(73) Assignee: Massachusetts Institute of Technology, Cambridge, MA (US)

(21) Appl. No.: 16/791,819

(22) Filed: Feb. 14, 2020

Related U.S. Application Data
(60) Provisional application No. 62/806,630, filed on Feb. 15, 2019.

(51) Int. Cl. A61K 31/4174; A61K 31/135; A61K 31/5513; A61K 31/05; A61K 33/16; A61K 9/00

(52) U.S. Cl. CPC A61K 31/4174; A61K 31/135; A61K 9/0019; A61K 31/05; A61K 33/16; A61K 31/5513

(57) ABSTRACT

Disclosed herein are methods and compositions for reversing the effects of a sedation agent or general anesthetic agents. The methods are useful for example for assessing a patient during a surgical procedure or a patient's physiologic state during and/or after a surgical procedure. Other uses for the methods and compositions include modulating a circadian rhythm, treating cardiac arrest, hypotension and bradycardia, and preventing emergence delirium.

### RELATED APPLICATIONS

This application claims priority under 35 U.S.C. § 119(e) to U.S. Provisional Application Ser. No. 62/806,630, filed on Feb. 15, 2019 which is herein incorporated by reference in it's entirety.

### FEDERALLY SPONSORED RESEARCH

This invention was made with Government support under Grant No. R01 MH115592 and R01 GM118269 awarded by the National Institutes of Health. The Government has certain rights in the invention.

### FIELD OF THE INVENTION

The invention relates in some aspects to an anesthesia- or sedation-reversing agent and methods for using these agents, for instance, to assess a surgical procedure or a patient's physiologic state during and/or after a surgical procedure.

### BACKGROUND

General anesthesia is widely used in clinical and research contexts. It is used to place patients in an insensate state during major surgery, so that surgeons may perform highly invasive, lengthy, and/or complex procedures. Some of general anesthesia's intended immediate effects are to induce analgesia, immobility, unconsciousness, and amnesia while keeping the patients physiology stable during a surgical procedure. Recovery of consciousness from the drug-induced anesthesia can be achieved by reducing or cessating the administration of the anesthetic drugs.

Lighter forms of general anesthesia are used for attenuating, specifically, the level of consciousness, without necessarily affecting a patient's mobility or analgesia. This state, referred to as sedation, is commonly utilized outside of operating rooms, such as in the intensive care units of hospitals.

### SUMMARY

The present disclosure is based, inter alia, on the discovery that an α2 adrenergic receptor antagonist, particularly atipamezole, can be used to rapidly reverse the effect of α2 adrenergic receptor agonists used in cases ranging from surgery to ICU and inpatient/ongoing hospital care, and the consequent ability to perform new medical treatments and procedures.

The present disclosure is based, inter alia, on methods for administering an α2 adrenergic receptor antagonist, particularly atipamezole, to a human under general anesthesia during a surgical procedure in order to induce a temporary and rapid reversal of unconsciousness (also referred to herein as a planned wakeup) and conduct an intraoperative assessment. Thereafter, the state of unconsciousness is restored for the completion of the surgical procedure.

Accordingly, one aspect of the present disclosure provides a method comprising administering to a subject an effective amount of an α2 adrenergic receptor agonist to perform a surgical procedure, inducing a planned wake-up by administering to the subject an effective amount of α2 adrenergic receptor antagonist (wherein the adrenergic receptor agonist is maintained or reduced), conducting an intraoperative assessment of the subject during the planned wakeup, and ceasing the α2 adrenergic receptor antagonist in order to proceed with the surgical procedure, and optionally administering to the subject an effective amount of a general anesthetic. In some embodiments, the intraoperative assessment is performed in an operating room. In some embodiments, after ceasing the α2 adrenergic receptor antagonist, the amount of the adrenergic receptor agonist is increased to its amount at the initiation of the surgical procedure. In some embodiments, the surgical procedure is a neurosurgery or an orthopedic surgery. In some embodiments, the neurosurgery is a craniotomy. In some embodiments, the orthopedic surgery is a spine surgery. In some embodiments, the intraoperative assessment is a physiologic or functional assessment of an organ, organ system, or anatomical region. In some embodiments, the intraoperative assessment is an evaluation of the surgical procedure. In some embodiments, the intraoperative assessment is to assess the subject's breathing. In some embodiments, the intraoperative assessment is a standard motor and language mapping of the subject's brain. In some embodiments, the intraoperative assessment is a standard motor mapping technique of the subject's spinal cord or musculature.

A further aspect of the present disclosure provides a method of administering to a subject undergoing a surgical procedure an effective amount of an α2 adrenergic receptor agonist to perform a surgical procedure, ceasing the administration of the α2 adrenergic receptor agonist and administering to the subject an effective amount of α2 adrenergic receptor antagonist at or near the end of the surgery, and conducting a post-operative assessment. In some embodiments, the post-operative assessment is conducted in the operating room after the end of the surgery. In some embodiments, the post-operative assessment is conducted in the intensive care unit (ICU) after the end of the surgery. In some embodiments, the post-operative assessment is conducted in the post-anesthesia care unit (PACU) after the end of the surgery. In some embodiments, the post-operative assessment is conducted in the surgical intensive care unit (SICU) after the end of the surgery. In some embodiments, the post-operative assessment is conducted within 30 minutes of the end of the surgery or within 1 hour of the end of the surgery or within 90 minutes of the end of the surgery. In some embodiments, the post-operative assessment is conducted within 10 minutes of the end of the surgery.

A further aspect of the present disclosure provides a method of administering to a subject undergoing a surgical procedure an effective amount of an α2 adrenergic receptor agonist to perform a surgical procedure, ceasing the administration of the α2 adrenergic receptor agonist and administering to the subject an effective amount of α2 adrenergic receptor antagonist at or near the end of the surgery, wherein the subject's mobility in an intensive or critical care unit is increased. In some embodiments, the subject's mobility is increased within 90 minutes of the end of the surgery or within 10-30 minutes of the end of the surgery.

A further aspect of the present disclosure provides a method of modifying circadian rhythms by administering to a subject in need thereof, treated with an α2 adrenergic agonist, an effective amount of an α2 adrenergic receptor antagonist in order to facilitate normal adaptation to regular day-light cycles or adjust the subject's circadian rhythms. In some embodiments, the subject is a patient at risk of, known to have, or diagnosed with delirium. In some embodiments, the α2 adrenergic receptor agonist is administered for sleep at night time or as desired. In some embodiments, the α2 adrenergic receptor antagonist is administered to restart a circadian rhythms cycle.

A further aspect of the present disclosure provides a method of treating cardiac arrest, the method comprising administering to a subject under general anesthesia and experiencing cardiac arrest, an effective amount of an α2 adrenergic receptor antagonist to treat the cardiac arrest, wherein the subject under general anesthesia had received an α2 adrenergic receptor agonist.

A further aspect of the present disclosure provides a method of treating bradycardia, the method comprising administering to a subject under general anesthesia and experiencing bradycardia, an effective amount of an α2 adrenergic receptor antagonist to treat the bradycardia, wherein the subject under general anesthesia had received an α2 adrenergic receptor agonist.

A further aspect of the present disclosure provides a method of treating hypotension, the method comprising administering to a subject under general anesthesia and experiencing hypotension, an effective amount of an α2 adrenergic receptor antagonist to treat the hypotension, wherein the subject under general anesthesia had received an α2 adrenergic receptor agonist.

A further aspect of the present disclosure provides a method of treating hypotension, the method comprising administering to a subject under sedation and experiencing hypotension, an effective amount of an α2 adrenergic receptor antagonist to treat the hypotension, wherein the subject under sedation had received an α2 adrenergic receptor agonist.

A further aspect of the present disclosure provides a method comprising administering to a subject an effective amount of an α2 adrenergic receptor agonist to perform a surgical procedure (wherein the subject is given an endotracheal breathing tube), ceasing the administration of the α2 adrenergic agonist, administering to the subject an effective amount of α2 adrenergic receptor antagonist prior to the end of the surgical procedure, and conducting an extubation when the subject displays a brainstem reflex. In some embodiments, prior to the end of the surgical procedure is between 0 and 15 minutes before the end of the surgical procedure. In some embodiments, the brainstem reflex is a pharyngeal reflex or a tracheal reflex.

A further aspect of the present disclosure provides a method comprising administering to a subject an effective amount of an α2 adrenergic receptor agonist to perform a surgical procedure, ceasing the administration of the α2 adrenergic agonist, administering to the subject an effective amount of α2 adrenergic receptor antagonist prior to the end (or at the end) of the surgical procedure, and assessing the subject for a brainstem reflex. In some embodiments, prior to the end of the end of the surgical procedure is between 0 and 15 minutes before the end of the surgical procedure. In some embodiments, the effective amount of α2 adrenergic receptor antagonist is administered after or at the end of the surgical procedure. In some embodiments, the effective amount of α2 adrenergic receptor antagonist is administered within 30 minutes after the end of the surgical procedure.

A further aspect of the present disclosure provides a method for reversing a neural blockade during a surgical procedure by administering to a subject undergoing a surgical procedure who has received a muscle blockade agent and a neural blockade agent, an effective amount of an α2 adrenergic receptor antagonist to reverse the neural blockade. In some embodiments, the method includes conducting an intraoperative assessment of the subject following the administration of the α2 adrenergic receptor antagonist. In some embodiments, the α2 adrenergic receptor antagonist is given in a dose range of 30:1-400:1 μg/kg α2 adrenergic receptor antagonist to neural blockade agent.

A further aspect of the present disclosure provides a method for reducing or preventing emergence delirium by administering to a subject under general anesthesia, an effective amount of an α2 adrenergic receptor antagonist for the subject to regain consciousness or to minimize symptoms of emergence delirium, wherein the subject under general anesthesia had received an α2 adrenergic receptor agonist. In some embodiments, the α2 adrenergic receptor antagonist is administered before emergence from general anesthesia. In some embodiments, the α2 adrenergic receptor antagonist is administered as the subject begins to regain consciousness. In some embodiments, the α2 adrenergic receptor antagonist is administered in an effective amount to stop at least one symptom of emergence delirium.

In further embodiments of the present disclosure, the α2 adrenergic receptor antagonist is administered in a dose range of 30:1-400:1 μg/kg α2 adrenergic receptor antagonist to agonist (e.g., α2 adrenergic agonist). In further embodiments of the present disclosure, the α2 adrenergic receptor antagonist provides rapid reversal of unconsciousness or sedation. In further embodiments, the α2 adrenergic receptor antagonist is atipamezole. In further embodiments, the α2 adrenergic receptor antagonist is administered intravenously. In further embodiments, the α2 adrenergic receptor antagonist is a composition comprising the α2 adrenergic receptor antagonist and a pharmaceutically acceptable excipient. In further embodiments, the pharmaceutically acceptable excipient is a saline or water-based solution. In further embodiments, the α2 adrenergic receptor antagonist is administered intravenously as a bolus or an infusion. In further embodiments, the α2 adrenergic receptor agonist is dexmedetomidine or clonidine. In further embodiments, the α2 adrenergic receptor agonist is administered in solo or in combination with other general anesthetics or sedative drugs. In further embodiments, the general anesthetic is propofol, sevoflurane, ketamine, or benzodiazepines. In further embodiments, the subject is a mammal. In further embodiments, the subject is a human.

The methods disclosed herein that apply to a subject under general anesthesia, wherein the subject under general anesthesia had received an α2 adrenergic receptor agonist, may also apply to methods for treating a subject under sedation, wherein the subject under sedation had received an α2 adrenergic receptor agonist.

These and other aspects of the invention, as well as various embodiments thereof, will become more apparent in reference to the detailed description of the invention. This invention is not limited in its application to the details of construction and the arrangement of components set forth in the following description or illustrated in the drawings.

### DETAILED DESCRIPTION

The disclosure provides, in certain aspects, methods of rapidly reversing the effects of general anesthesia or sedation by administering an α2 adrenergic receptor antagonist. In a preferred embodiment the α2 adrenergic receptor antagonist is atipamezole. Current drugs used in anesthesia and sedation typically require the subject be intubated and rely on reducing or stopping administration of the drug in order for the patient to regain consciousness, which can be a lengthy process.

Herein, the present disclosure teaches an application for atipamezole in human surgical procedures that has not been used previously and has the potential to shift the paradigm of surgical medicine. Currently, there is no clinical use of atipamezole in humans. The present disclosure teaches methods for administering atipamezole to a subject (e.g., patient, human, etc.) during a surgical procedure in order to conduct a planned wakeup (described further below) and evaluate the subject in the middle (or prior to the end) of the surgical procedure. Atipamezole induces a rapid reversal of unconsciousness (or sedation), thus resulting in the planned wakeup.

Currently, in surgical cases in the operating room, there are no effective methods for the temporary reversal of unconsciousness in human patients, which would allow a clinician to make early (intraoperative) assessments/diagnoses. Clinicians have to wait until after the surgical procedure—after the effects of the general anesthesia have subsided—to assess the patient. Examples of the assessments include checking the patient's motor function and neurological function, cognitive exams, etc. The methods described in the present application would allow an intraoperative assessment of the patient, a determination of how the surgical technique is impacting the patient during surgery, the identification of surgical errors that would impact, for example, motor function and neurological function, and the correction of surgical errors in real-time thus minimizing the requirement for a second (separate) corrective surgical procedure. These would substantially improve surgical outcomes and open up possibilities for new, currently unknown and/or unpracticed surgical procedures. In a preferred embodiment, the α2 adrenergic receptor antagonist, atipamezole, is administered intravenously.

Often in neurosurgical and orthopedic surgery cases, for example, the subject will have received general anesthesia in standard current practices, and surgery will be performed to expose a region of the central nervous system such as the brain or spinal cord. In some embodiments, before surgery can proceed on the sensitive region of interest, the subject can be re-awakened by administering an α2 adrenergic receptor antagonist (e.g., atipamezole) for an intraoperative assessment of nervous system function. This is done in order to determine the functions of the regions of potential surgical intervention in order to avoid damaging regions of critical importance (e.g., avoiding critical language regions in the brain during tumor removal via patient's speaking, ensuring spinal cord integrity by voluntary movements). In contrast, current practice involves turning down the levels of anesthetic drugs and waiting for them to wear off once the surgical site has been exposed and before the surgery can proceed.

It has been discovered herein that the effects of general anesthesia and sedation agents used alone or in combination can be predictably and efficiently reduced through use of an α2 adrenergic receptor antagonist, as described herein. The α2 adrenergic receptor antagonist is sufficient to reverse neurological sedation in an anesthetized/sedated patient in a manner that enables a physician to conduct intra-operative assessments on the patient that was under general anesthesia. The α2 adrenergic receptor antagonists are particularly useful in surgical procedures where a patient has received a full anesthetic treatment including a muscle blockade agent and a neural blockade agent. The α2 adrenergic receptor antagonist is sufficient to reverse the neural blockade and enable the patient to communicate with the surgical team and or regain sufficient cognitive function in order to function at a normal level. In alternative embodiments, rather than waiting for the anesthesic to wear off, the α2 adrenergic receptor antagonist can be administered immediately following an intensive procedure where the patient was under general anesthesia. The rapid reversal of the unconsciousness would allow assessment (e.g., of patient mobility, language skills, cognitive ability) much sooner than previously possible. This can be conducted prior to the transfer of the patient from the operating room to the post-surgical inpatient hospital unit.

Previous studies have shown that about 15% of patients have failed extubations. The high rates of failed extubations lead to unnecessary (and costly) prolongation of intubation in hospitals. The present disclosure teaches a method of using the α2 adrenergic receptor antagonist (e.g., atipamezole) to promote spontaneous breathing or promote brainstem reflexes in a subject that was under general anesthesia. In some embodiments a subject is administered α2 adrenergic receptor agonist and is intubated. Prior to the end of the surgical procedure (e.g. 10 minutes prior), the subject is administered an effective amount of the α2 adrenergic receptor antagonist, which promotes (i.e., results in rapid onset of) breathing or promotes a pharyngeal reflex (i.e. gag-reflex). Upon observation of the breathing or the pharyngeal reflex, the subject is extubated. Typically, when a patient is intubated and under general anesthesia, the clinician waits for the subject to start having a gag-reflex, as an indication that the subject has recovered spontaneous breathing, before pulling out the endotracheal breathing tube. Administering atipamezole could accelerate this process. In instances when the subject does not have a gag-reflex towards the end or after the surgery, the clinicians conduct a trial extubation. Here, the use of atipamezole would reduce or obviate the need for trial extubation and lead to new surgical protocols that better facilitate the recovery of breathing.

It has also been discovered herein that α2 adrenergic receptor antagonists may be used to regulate circadian rhythms. In patients that need assistance in regulating circadian rhythms α2 adrenergic receptor antagonists may be delivered alone or in a cyclic combination with an α2 adrenergic receptor agonist in order to facilitate normal adaptation to regular day-light cycles or adjust the subject's circadian rhythms.

The α2 adrenergic receptor antagonists may also be used to treat cardiac arrest occurring while a patient is under general anesthesia. The patient who has gone into cardiac arrest can be administered an α2 adrenergic receptor antagonist to reverse the sedative effects or effects of general anesthesia and enable or support the restoration of cardiac activity in the patient.

### General Anesthesia and Sedation

General anesthesia is a drug-induced comatose state resulting from the administration of one or more general anesthetic agents and is characterized by a loss of consciousness. Typically, the induction of general anesthesia includes a balance of potent sedatives, muscle relaxants, opioids, neuromuscular blocking agents, and local anesthetics. General anesthesia is widely used in the medical field for highly invasive, lengthy, and/or complex procedures. In addition to unconsciousness, other effects of general anesthesia include amnesia, analgesia, antinociception, akinesia, paralysis, and physiological stability. Analgesia or nociceptive blockage (or antinociception) prevents central nervous system arousal and cardiovascular and neurohumoral responses to surgery (Sanders et al., 2012). Nociceptive blockage refers to the blockage of pain caused by tissue damage. In standard practice, following a major surgery with general anesthesia, the administration of a general anesthetic is typically reduced or terminated to allow the subject (e.g., patient) to regain consciousness. This is a slow, suboptimal process. In current practice, most of the agents used for the post-operative reversal of the general anesthetics are centered on the use of muscle relaxants. Disclosed herein are methods for accelerating this process and, most importantly, allowing a subject to regain cognitive faculties rapidly through the use of a reversal agent.

The term “general anesthetic” refers to a drug that induces general anesthesia, as defined herein. Non-limiting examples of general anesthetics include nitrous oxide, sevoflurane, halothane, xenon, enflurane, chloroform, isoflurane, methoxyflurane, desflurane, ethyl chloride, cyclopropane, chloral hydrate, ketamine, esketamine, etomidate, propofol, chlorobutanol, guanabenz, guanfacine, clonidine, tizanidine, medetomidine, and dexmedetomidine.

The term “sedation,” as used herein, refers to a rousable state or depression in consciousness caused by sedative drugs. In standard practice, sedation is typically used for minor surgical interventions. During sedation, a subject shows decreased arousal or consciousness, wherein consciousness can be described as an awareness of self and environment or the awareness of external stimuli. The effects of sedation include anxiolysis, and/or amnesia, and/or analgesia. The term “sedative drugs” or “sedatives” encompasses a broad spectrum of drugs that slow normal brain function. They have different mechanisms of action, but mainly function by increasing the neurotransmitter gamma-aminobutyric acid (GABA), which in turn depresses the central nervous system (CNS). Types of sedatives include barbiturates, benzodiazepines, nonbenzodiazepine hypnotics, first generation antihistamines, herbal sedatives, methaqualone (and analogues), muscle relaxants, opioids, antidepressants, and antipsychotics.

Examples of barbiturates include Amobarbital, Aprobarbital, Butabarbital, Mephobarbital, Methohexital, Pentobarbital, Phenobarbital, Primidone, Secobarbital, and Thiopental. Examples of benzodiazepines include Alcohol (ethyl alcohol or ethanol), Alprazolam, Chloral hydrate, Chlordiazepoxide, Clorazepate, Clonazepam, Diazepam, Estazolam, Flunitrazepam, Flurazepam, Lorazepam, Midazolam, Nitrazepam, Oxazepam, Temazepam, and Triazolam. Examples of nonbenzodiazepine hypnotics include Eszopiclone, Zaleplon, Zolpidem, and Zopiclone. Examples of first generation antihistamines include Diphenhydramine, Dimenhydrinate, Doxylamine, Promethazine, Hydroxyzine, Brompheniramine, and Chlorpheniramine. Examples of herbal sedatives include Duboisia hopwoodii, chamomile, Prostanthera striatiflora, catnip, Piper methysticum, valerian, cannabis, Passiflora incarnata, Physochlaina, and menthyl isovalerate. Examples of methaqualone (and analogues) include Afloqualone, Cloroqualone, Diproqualone, Etaqualone, Methaqualone, Methylmethaqualone, Mebroqualone, Mecloqualone, and Nitromethaqualone. Examples of muscle relaxants include Baclofen, Meprobamate, Carisoprodol, Cyclobenzaprine, Metaxalone, Methocarbamol, Tizanidine, Chlorzoxazone, Orphenadrine, Gabapentin, and Pregabalin. Examples of opioids include Tramadol, Tapentadol, Morphine, Hydromorphone, Oxymorphone, Oxycodone, Hydrocodone, Methadone, Propoxyphene, Meperidine, Fentanyl, Codeine, Carfentanil, Remifentanil, Alfentanil, Sufentanil, and Opium. Examples of antidepressants include Amitriptyline, Trazodone, Mirtazapine, Doxepin, Desipramine, Imipramine, Clomipramine, Amoxapine, Trimipramine, Nortriptyline, and Nefazodone. Examples of antipsychotics include Olanzapine, Clozapine, Thiothixene, Haloperidol, Fluphenazine, Prochlorperazine, Trifluoperazine, Loxapine, Quetiapine, and Asenapine.

Medication to induce general anesthesia or sedation can be administered, for example, through a breathing mask or tube, or given as an injection. The injection can be intravenously, intradermally, intraarterially, intralesionally, intratumorally, intracranially, intraarticularly, intraprostaticaly, intrapleurally, intratracheally, intravitreally, intravaginally, intrarectally, topically, intratumorally, intramuscularly, intraperitoneally, subcutaneously, subconjunctival, intravesicularlly, mucosally, intrapericardially, intraumbilically, and/or intraocularally. In some embodiments, the medication used to induce general anesthesia or sedation can also be administered by infusion, such as intravenous infusion.

General anesthesia and sedation are dose-dependent and there exists a lot of person-to-person variation in response to doses.

In some embodiments, a subject is placed under general anesthesia with a general anesthetic such as propofol, a rapid-activating GABAergic drug that can cause cessation of breathing and require intubation. For medical evaluations, the general anesthetic is turned off until the subject reaches the desired state of consciousness and then turned back on after the evaluation. In some embodiments of this disclosure, with the use of alternative sedatives, such as α2 adrenergic receptor agonists, either alone or in combination with general anesthetics, it is possible to administer an anesthesia-reversing agent to rapidly reverse the effects of the sedative so that the subject's medical evaluation can be carried intraoperatively and more efficiently.

### Anesthesia-Reversing Agents

Described herein, in some aspects, are methods for administering an anesthesia-reversing agent to a subject who is under general anesthesia or sedation. Anesthesia-reversing agents (e.g., reversal agents), as used herein, reverse the effects of anesthetic drugs or sedative drugs. There are two main types of anesthesia-reversing drugs: receptor-specific antagonists and non-specific analeptic agents. Receptor-specific antagonists, used in the present disclosure, have no intrinsic capacity for anesthesia reversal but have a high affinity for a receptor involved in the anesthetic effect pathway. Non-limiting examples of such receptor-specific antagonist include atipamezole, anticholinesterases, naloxone, flumazenil, nalmefene, and naltrexone (Pani et al., 2015).

In several major surgical procedures, it would be useful to wake a subject during the surgery. This practice is referred to as a “planned wake-up”. In some embodiments of the disclosure, the subject may be returned to a certain level of consciousness during the procedure to evaluate the subject's physiology (e.g., the effectiveness of the treatment or intervention). In yet another embodiment of the disclosure, the subject may be returned to a certain level of consciousness during the procedure in response to an emergency situation. Currently, the standard practice is to reduce or terminate the administration of the anesthetic drug or sedative, and wait for its effects to wear off, which is an impractical solution due to loss of time and space in the operating room. In emergency situations, this practice also increases the risk of secondary complications and/or death to the subject. This practice can have additional adverse effects on the subject: for example, a slow and gradual emergence from unconsciousness or sedation can be disorienting and has been attributed to cases of delirium, post-surgery. Thus, there is a dire need for improved methods to induce rapid reversal of (general anesthesia-related) unconsciousness or sedation, in order to conduct intra-operative or post-operative assessments of a patient's physiology, evaluate treatments or surgical procedures, and improve patient outcomes.

As used herein, the term “emergence” refers to the process of regaining consciousness. This is a process during which a subject regains cognitive and, optionally motor faculties. As used herein, the term “planned wake-up” refers to the process of intentionally inducing consciousness and the return of cognitive faculties in a subject during a surgical procedure. In some embodiments, this planned wake-up is for purposes of an assessment. In alternative embodiments, this planned wake-up is performed in response to an emergency during the surgery.

Typically, the administration of anesthetics and sedatives is terminated to facilitate arousal or emergence from unconsciousness or sedation, which can take a prolonged period of time and render planning the end or subsequent step of a procedure difficult. Subjects typically do not regain cognitive and/or motor faculties quickly when anesthetic administration is decreased or stopped, which may delay extubation of a patient and thus require intensive care unit (ICU) admission. In order to reduce the time to emergence, additional drugs (i.e., anesthesia-reversing agents) can be administered during or at the end of a procedure.

As used herein, the terms “intensive care unit” and “critical care unit” are used interchangeably. ICU, as used herein, refers to a ward in which subjects (e.g., hospital patients) are cared for. These subjects are in critical condition, have undergone major surgery, and are under constant observation. Subjects are never discharged from the ICU, and instead are moved to standard medical hospital floors once stable.

### α2 Adrenergic Receptor Agonists

As described above, there are a number of general anesthetics and sedatives. Of particular interest to the present disclosure are α2 adrenergic receptor agonists (also known as α2 adrenoceptor agonists). These agonists are widely used as anesthesia adjuncts and, less commonly, used alone (i.e. in solo). As used herein, the term “adjunct” refers to an agent that is used in combination (i.e. as a supplement) and, in the context of this disclosure, enhances or augments anesthetic effects.

One of the reasons for the broad use of α2 adrenergic receptor agonists as anesthesia adjuncts in the medical field is minimalization of a subject's requirement for additional sedative or anesthetics. Many of the additional surgical anesthetics are not tolerated well, especially in children and the elderly, which makes surgery and sedation less safe. Some of the associated risks with these drugs include, but are not limited to, acute hypertension, rebound hypertension after cessation of the anesthetic or sedative, nausea, delirium after cessation of the anesthetic or sedative, hepatoxicity, elevated serum concentrations, bradycardia, tachyphylaxis, hypoxia, and atrial fibrillation.

An α2 adrenergic receptor inhibits norepinephrine release from the presynaptic neuron. This is accomplished through the inhibition of adenylate cyclase, which decreases the formation of 3,5-cyclic adenosine monophosphate (cAMP). Through modulation of calcium-activated activity, specifically potassium efflux, calcium entry into the nerve channel is blocked and the membrane hyperpolarizes. Norepinephrine secretion and neuronal firing are, in turn, suppressed (Gertler, 2001; Giovannitti, 2015). Non-limiting examples of α2 adrenergic receptor agonists include guanabenz, guanfacine, clonidine, tizanidine, medetomidine, and dexmedetomidine.

Dexmedetomidine (e.g., PRECEDEX™ and DEXDOMITOR®) is a prototypical central α2 adrenergic receptor agonist, a sedative, and has been utilized most broadly in veterinary contexts. It is prescribed for intravenous use. DEXDOMITOR® (dexmedetomidine hydrochloride) is indicated for use in dogs and cats, while PRECEDEX™ (dexmedetomidine hydrochloride) is indicated for use in humans. Dexmedetomidine is an imidazole compound and an active dextroisomer of medetomidine. It has a highly selective affinity for the α2 adrenoceptor and selectively binds to presynaptic α2 adrenoceptor, which inhibits the post-synaptic activation of adrenoceptors, suppresses sympathetic activity, and induces sedation and anxiolysis.

Compared to other more commonly-used anesthetics and sedatives, dexmedetomidine is a safer option that, when supplemented to current anesthesia cocktails, has been shown to dramatically reduce the quantity of other surgical anesthetics required for general anesthesia and sedation. It can be administered orally, transmucosally, transdermally, intravenously, or intramuscularly. When delivered by intravenous (IV) infusion at low doses, it decreases cardiac output and systolic blood pressure. Unlike other anesthetics and sedatives, dexmedetomidine has a minimal effect on the respiratory system and can provide adequate rousable sedation without intubation (Giovannitti, 2015).

In previous years, dexmedetomidine was not preferred for human clinical use, primarily due to its terminal elimination half-life of about 2 hours—its prolonged efficacy. It was primarily used for non-human subjects, including, but not limited to monkeys, dogs, and pigs. However, despite its prolonged duration of efficacy, dexmedetomidine is increasingly being used in more human surgeries due to the aforementioned advantages it has over other anesthetic and sedative drugs. Dexmedetomidine is currently approved by the United Stated Food and Drug Administration (FDA) for sedation of initially intubated and mechanically ventilated patients in an intensive care setting and sedation of non-intubated patients prior to and/or during surgical procedures. Methods for administering dexmedetomidine to patients in an intensive care unit are provided in U.S. Pat. No. 6,716,867.

Clonidine (e.g., CATAPRES® and KAPVAY™) is an imidazoline derivative and a centrally-acting α2 adrenergic receptor agonist. Clonidine binds to a central α2 adrenergic receptor, thus suppressing norepinephrine release and decreasing the sympathetic outflow to the heart, kidneys, and peripheral vasculature. In doing so, clonidine reduces the blood pressure and heart rate of a subject. Clonidine is used as a sedative and analgesic. It reduces post-operative shivering. Its additional uses are for the treatment of hypertension, ADHD, and cancer pain in human subjects. It has also been used for prophylaxis of vascular migraine headaches, treatment of severe dysmenorrhea, management of vasomotor symptoms associated with menopause, and treatment of opiate, benzodiazepine, alcohol, cocaine, food, and tobacco withdrawal. It is prescribed for oral use and has an elimination half-life ranging from 12 hours to 16 hours. Clonidine has resulted in unpredictable hemodynamic effects (e.g., hypotension and tachycardia), which has limited its use on critically ill subjects. (Tryba et al, 1993)

In some aspects, the disclosure provides a means to counter the prolonged efficacy of these α2 adrenergic receptor agonists/sedatives through the use of an anesthesia-reversing agent, for example, an α2 adrenergic receptor antagonist.

### α2 Adrenergic Receptor Antagonists

An α2 adrenergic receptor antagonist, as used herein, is a neural blockade reversing agent that specifically antagonizes α2 adrenergic receptors. α2 adrenergic receptor antagonists are known in the art and include but are not limited to Atipamezole, Aripiprazole, Asenapine, Cirazoline, Clozapine, Efaroxan, Idazoxan, Lurasidone, Melperone, Mianserin, Mirtazapine, Napitane, Olanzapine, Paliperidone, Phenoxybenzamine, Phentolamine, Piribedil, Rauwolscine, Risperidone, Rotigotine, Quetiapine, Norquetiapine, Tetiptiline, Tolazoline, Yohimbine, and Ziprasidone.

Atipamezole (MPV-1248) or 4-(2-ethyl-2,3-dihydro-1H-inden-2-yl)-1H-imidazole] (ANTISEDAN®) is a synthetic agent with an imidazole structure. It has the following chemical structure:

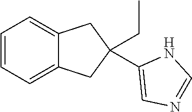 [SMILES: CCC1(c2cnc[nH]2)Cc2ccccc2C1]

In some embodiments, the methods of the invention use atipamezole, a synthetic α2 adrenergic receptor antagonist, to rapidly reverse the sedative and analgesic effects of α2 adrenergic receptor agonists, such as dexmedetomidine, medetomidine, romifidine, and xylazine. Atipamezole has been used in veterinary medicine. It is currently administered to dogs as an intramuscular injection. Clinically, it has not been used for human surgeries.

After administration, atipamezole rapidly crosses the blood brain barrier. It is highly selective for α2-adrenergic receptors, which are in the central synapses and are located presynaptically and/or postsynaptically. There are three types of α2-adrenergic receptors, α2A, α2B, and α2C. α2A are located on the locus coeruleus, brainstem, cerebral cortex, septum, hypothalamus, hippocampus and amygdala. α2B is located in the thalamus, and α2C is located in the basal ganglia, olfactory tubercle, hippocampus, and cerebral cortex (Scheinin, 1994). Atipamezole competitively displaces α2-adrenergic receptor agonists. In addition, it has no affinity for other receptors, which increases its effectiveness and minimizes adverse effects from the drug. Upon administration, atipamezole has been shown to reverse sedation, increase subject mobility after a non-invasive or minor surgery, and reverse the cardiovascular effects (e.g., bradycardia) of α2-adrenergic receptor agonists (e.g., dexmedetomidine and clonidine). In certain cases, administration of atipamezole results in motor restlessness, hypotension, and/or tachycardia, and thus it has not been recommended for intravenous administration (Bahri, 2008).

Atipamezole has a rapid onset, at which point a subject under sedation regains consciousness and motor and/or cognitive faculties. Intramuscular injection of atipamezole has been shown to reverse the effects of dexmedetomidine (e.g., DEXDOMITOR and DEXDOMITOR 0.1) in 5-10 minutes. As used herein, the term “rapid” or “rapidly” refers to an onset ranging from 1 minute to 20 minutes following the administration of the α2 adrenergic receptor antagonist. According to some aspects of the present disclosure, rapid onset of α2 adrenergic receptor antagonist is 1, 2, 3, 4, 5, 6, 7, 8, 9, 10, 11, 12, 13, 14, 15, 16, 17, 18, 19, or 20 minutes.

The use of atipamezole or other α2 adrenergic receptor antagonists as reversal agents would expand the use of α2 adrenergic receptor agonists such as dexmedetomidine in operating rooms and ICUs and allow subjects to emerge from unconsciousness more rapidly, enabling intraoperative evaluations, post-operative evaluations, and quick discharge. This new application for atipamezole could radically transform current practice and open up possibilities for unknown procedures and treatments in future.

### Intraoperative and Post-Operative Surgery-Related Use of α2 Adrenergic Receptor Antagonist

In surgical cases in the operating room, a temporary reversal of unconsciousness may be desired. Often in neurosurgical and orthopedic surgery cases, for example, the subject will have received general anesthesia in standard current practices and surgery will be performed to expose a region of the central nervous system such as the brain or spinal cord. In some embodiments, before surgery can proceed on the sensitive region of interest, the subject will be re-awakened by administering an α2 adrenergic receptor (e.g., atipamezole) for an intraoperative assessment of nervous system function. This is done in order to determine the functions of the regions of potential surgical intervention in order to avoid damaging regions of critical importance (e.g., avoiding critical language regions in the brain during tumor removal via patient's speaking, ensuring spinal cord integrity by voluntary movements). In contrast, current practice involves turning down the levels of anesthetic drugs and waiting for them to wear off once the surgical site has been exposed and before the surgery can proceed.

In some aspects of the present disclosure, a subject treated with general anesthetic agents, including an α2 adrenergic receptor agonist, will undergo the initial stages of relevant surgical procedures. Upon the clinician's decision to evaluate the functions and/or physiologic status of an organ, organ system, or anatomical region of importance to the clinicians, administration of an α2 adrenergic receptor antagonist will be carried out as either a single bolus, multiple boluses, or as a continuous infusion. At this time (or prior to or following this administration), the standard anesthetic protocol for facilitating reversal of intraoperative sedation may be begun or the pre-reversal protocol may be maintained. Part way through or following the conclusion of the physiologic or functional assessment, intravenous α2 adrenergic receptor antagonist will cease infusion, and the surgical procedure will proceed following current practices with re-initiation of general anesthesia, after a planned wake-up.

In some embodiments of the present disclosure, an α2 adrenergic receptor antagonist is used for rapid reversal of unconsciousness in an awake craniotomy, whereby a subject is administered an α2 adrenergic receptor agonist (e.g., dexmedetomidine) and is fully unconscious under general anesthesia. The subject is administered an α2 adrenergic receptor antagonist to regain cognitive faculties (while standard anesthetics remain on or are reduced prior to infusion), and standard motor and language mapping of the brain are carried out. The surgery proceeds as normal because the subject is fully anesthetized following intraoperative cognitive or physiological testing.

In some embodiments of the present disclosure, an α2 adrenergic receptor antagonist is used for rapid reversal of unconsciousness in a neurosurgery or orthopedic spine surgery, whereby a subject is administered an α2 adrenergic receptor agonist (e.g., dexmedetomidine) and is fully unconscious under general anesthesia. The subject is administered an α2 adrenergic receptor antagonist to regain active (awake) or improve passive/conductive motor faculties (while standard anesthetics remain on or are reduced prior to infusion), and standard motor mapping techniques of the spinal cord and musculature carried out. The surgery proceeds as normal because the subject is fully anesthetized following intraoperative testing.

Often in neurosurgical and orthopedic surgery cases, for example, the subject will have received general anesthesia in standard current practices and surgery will be performed on sensitive regions of interest within or approximating structures of the central or peripheral nervous system. It is of critical importance to know if the intervention succeeded or caused unacceptable side effects (e.g., in surgery on the spine that approximates the spinal cord, it is essential to know if the surgery placed pressure on the nerves and was blocking motor and sensory functions). Current practice involves turning down the levels of anesthetic drugs and waiting for them to wear off and allowing the patient to begin the wake-up process and be transferred to the post-operative care unit or a critical care unit. Often here, in these sites outside of the operating room, the first exams are done on the patients to evaluate the presence or absence of side-effects and overall effectiveness resulting from the surgery. In some embodiments, a subject is treated with general anesthetic agents, including the addition of an α2 adrenergic receptor agonist, and will undergo the initial stages of relevant surgical procedures. Prior to or upon conclusion of the surgical procedure, general anesthetic doses are reduced to help facilitate awakening of the subject. At these times, an α2 adrenergic receptor antagonist will be administered as either a single bolus, multiple boluses, or as a continuous infusion. The intended effect of the drug is to wake up the subject rapidly from a state of unconsciousness such that a clinical exam can be carried out in the operating room. Before the subject is relocated to the main hospital floor, the rapid arousal will allow the surgeons or additional medical clinical team members to evaluate the functions and/or physiologic status of an organ, organ system, or anatomical region of relevance to the surgical procedure previously completed. This is a critical diagnostic maneuver. This will allow for immediate evaluation of the subject's physiological state post-surgery. This rapid induction of wake-up may allow immediate diagnosis of potentially untoward events that can require further diagnostic evaluation and/or treatment. An anesthesia-reversal agent such as an α2 adrenergic receptor antagonist could open the possibility of evaluating the surgeries effectiveness still in the operating room because the subject will be sufficiently awake for clinical exams. This could radically change the practice of care, for example, as surgeries could be re-initiated in the operating room immediately to rectify otherwise devastating side effects, without reinitiating a whole new surgery at a different time.

The intensive care unit (ICU) receives patients following major trauma health events and following majorly invasive or complicated surgeries. In the ICU, human patients are often sedated with propofol, a rapid-acting GABAergic drug that can stop breathing and thus requires patients to be intubated. Patients in the ICU are continuously observed and receive regular health exams from the healthcare team to evaluate progress of symptoms and recovery of physiological functions. Currently, propofol infusions are scheduled to be turned off prior to these clinical exams. Dexmedetomidine, however, has shown promise in trials that α2-adrenergic drugs can be used in the critical care setting for sedation without the need for intubation. The prolonged duration of action of this dexmedetomidine make it extremely difficult, if not impossible, to efficiently schedule and perform clinical exams on patients requiring them. Administration of an α2 adrenergic receptor antagonist such as atipamezole would allow for rapid reversal of α2 adrenergic receptor agonist (e.g., dexmedetomidine) in the ICU, and allow clinical teams to evaluate a subject's physiologic functions efficiently and more safely.

In some aspects of the present disclosure, subjects in an intensive or critical care unit (e.g. medical ICU, post-surgical ICU, cardiac ICU, neurological ICU) receive α2 adrenergic receptor agonists such as dexmedetomidine or clonidine with or without supplementation of other general anesthetics, such as propofol, sevoflurane, ketamine, or benzodiazepines. During their inpatient stay under continuous monitoring, the addition of an α2 adrenergic receptor antagonist, such as atipamezole, is used to reverse the sedative effects of the anesthetic drugs, specifically such that physical and physiological assessments can take place unencumbered. Once the assessment has taken place, next steps in treatment can be more accurately planned and a decision to restore or remove sedation from the patient can be made.

In some embodiments, the α2 adrenergic receptor antagonist (e.g., atipamezol) is administered with a second anesthesia-reversing agent. Non-limiting examples of anesthesia-reversing agents include neuromuscular blockade reversal agents (anticholinesterases, cyclodextrins, cysteine, 4-aminopyridine, galanthamine, suramin, etc.), opioid antagonists (naloxone, nalmefene, naltrexone etc.), sedative reversal agents (flumazenil, atipamezole, etc.), analeptic agents (doxapram, nikethamide, pentylenetetrazol, bemegride, amphetamine, methylphenidate, etc.), and anticoagulant reversal agents (e.g., platelet factor 4).

As used herein, the term “inpatient” refers to a setting wherein the patient is admitted and currently staying in the hospital. An inpatient facility is for patients that have not been discharged.

In some embodiments, a subject suffering from a traumatic injury and requiring a clinical exam in the ICU receives α2 adrenergic receptor agonist (e.g., dexmedetomidine) sedation with or without other anesthetic drugs. Sedative and anesthetic agents administered to subjects are either maintained or changed at the time of the clinical exam. An α2 adrenergic receptor antagonists, for example, are administered intravenously either as a bolus, multiple boluses, or an infusion in order to reverse sedation sufficiently such that a clinical exam can be initiated. Following the exam or at the cessation of its attempt, standard ICU sedation protocols restart at the discretion of the physician based on the new clinical findings.

An alternative embodiment is an intubated subject that has suppressed breathing and is in the ICU receiving adrenergic receptor agonist (e.g., dexmedetomidine) sedation with or without other anesthetic drugs. In order to assess the viability of that subject's breathing abilities, intravenous α2 adrenergic receptor antagonist is administered via bolus, multiple boluses, or steady infusion. Spontaneous breathing is assessed following the administration of the α2 adrenergic receptor antagonist, and a new clinical decision is able to be made at this time to discern if extubation if indicated or sedation should be reinstated.

An alternative embodiment is the use of an α2 adrenergic receptor antagonist on subjects in the ICU following major surgery to improve long-term patient outcomes. An α2 adrenergic receptor antagonist is administered via bolus, multiple boluses, or steady infusion in order to get these subjects ambulatory. Reduction in sedation increases the time the subjects will spend walking and moving around during their stay, and will improve the chances for a better health outcome.

The term surgery (e.g., surgical procedure) refers to the use of a manual or instrumental technique on a subject to diagnose or treat a pathological condition, to improve a physiologic condition, or to enhance the appearance of an organ, organ system, or anatomical region. It includes any procedure (e.g., laparoscopic or otherwise requiring suturing or wound closure). Surgery can be performed by a doctor, surgeon, veterinarian, or dentist and can be performed in a hospital, veterinary clinic, dentist's office, or an equivalent health care facility. Surgery can also be performed bedside in an ICU/PACU/Surgical ICU, as emergencies arise, and make-shift operating areas are established at the bedside to deal with the urgent nature of the matter. The term “major surgery”, as used herein, refers to an invasive surgery for which the use of sedation or general anesthesia is recommended.

In some embodiments, the surgery is performed in an operating room (OR). The term “operating room” as used herein refers to a room within a hospital, veterinary clinic, or health care facility in which surgical procedures are performed under sterile conditions. To maintain the sterility of the operating room all the individuals wear personal protective equipment and have their nose and mouth covered, with the exception, in some instances, of the patient.

As used herein, the “end of the surgery” (or end of the surgical procedure) refers to the time of the assumed final role of the individual conducting the surgery (e.g., the surgeon, dentist, etc.) The end of the surgical procedure can refer to the point when the anesthesiologist takes over the surgery for extubation, the point when extubation has been completed (or successfully completed), the transfer of patient out of the operating room, the point of final wound closure (sutures set), the final surgical evaluation in the operating room, etc. In some embodiments, the end of the surgery can be defined as the completion time of the last medical procedure (e.g., billable medical procedure) carried out by a member of the surgical team before the transfer of acute management to a member of another staff (anesthesiologists, medical ICU docs, etc.). A person of ordinary skill in the art would recognize the end of the surgery (surgical procedure).

As used herein, the terms “subject” and “patient” are used interchangeably. In some embodiments, subject refers to a human, in other embodiments subject refers to a mammal, wherein the mammal is selected from a group including but not limited to non-human primate, cow, horse, pig, sheep, goat, dog, cat, rabbit, ferret, hippopotamus, giraffe, okapi, or rodent. In further embodiments, subject refers to a reptile, wherein the reptile is selected from a group including tortoises, turtles, alligators, and armadillos.

In some embodiments, an α2 adrenergic receptor antagonist is administered to a subject undergoing day-stay surgery, wherein “day-stay surgery” refers to surgery for more minor procedure, wherein the subject is discharged the same day of the surgery, generally without ICU admission. In some embodiments, an α2 adrenergic receptor antagonist is administered to a subject undergoing major surgery. In further embodiments, an α2 adrenergic receptor antagonist is administered to a subject that is undergoing a surgical procedure and will not be discharged on the same-day.

A surgical procedure, as used herein, includes but is not limited to a neurosurgery, orthopedic surgery, thoracic or cardiovascular surgery, gynecologic surgery, ophthalmologic surgery, oral or maxillofacial surgery, otolaryngological surgery, urological surgery, and cosmetic surgery. Non-limiting examples of neurosurgeries include spinal fusion, ventriculostomy, craniotomy, cranioplasty, decompressive craniectomy, trepanning, pallidotomy, anterior temporal lobectomy, thalamotomy, hemispherectomy, endoscopic thoracic sympathectomy, sympathectomy, bilateral cingulotomy, and lobotomy. Non-limiting examples of orthopedic surgery include spine surgery, scoliosis surgery, hand surgery, shoulder surgery, elbow surgery, total joint reconstruction (arthroplasty), skull reconstruction, foot surgery, and ankle surgery. Non-limiting examples of thoracic or cardiovascular surgery include Angioplasty, coronary artery bypass surgery, Valvuloplasty, Pericardiectomy, Endarterectomy, Cardiotomy, Thoracotomy, Pericardiotomy, Pneumonectomy, Pleurodesis, heart transplantation, and lung transplantation. Non-limiting examples of gynecologic surgery include Vaginoplasty, Clitoroplasty, Labiaplasty, Tuboplasty, Fimbrioplasty, Cervicectomy, Clitoridectomy, Oophorectomy, Salpingoophorectomy, Salpingectomy, Hysterectomy, Vaginectomy, Vulvectomy, Salpingostomy, Amniotomy, Clitoridotomy, Hysterotomy, Hymenotomy, Episiotomy, Symphysiotomy, Tubal ligation, Tubal reversal, Colporrhaphy, Cesarean section, Hymenorrhaphy, and Endometrial biopsy. Non-limiting examples of ophthalmologic surgery include Punctoplasty, Trabeculoplasty, Photorefractive keratectomy, Trabeculectomy, Iridectomy, Vitrectomy, Dacryocystorhinostomy, Radial keratotomy, Mini Asymmetric Radial Keratotomy, and Corneal transplantation. Non-limiting examples of oral or maxillofacial surgery include Tracheal intubation, Distraction osteogenesis, Cranioplasty, Sling, Labial frenectomy, Jaw reduction, Microsurgery, Genioglos sus advancement, Osteotomy, Bone grafting, Flap, Cheiloplasty, Maxillomandibular advancement, Facial feminization surgery, Face transplant, and Forehead lift. Non-limiting examples of otolaryngological surgery include insertion of grommets for glue ear, tonsillectomy, septoplasty, microlaryngoscopy, oesophagoscopy, endoscopic sinus surgery, tympanomastoid surgery, and tracheostomy. Non-limiting examples of urological surgery include Urethroplasty, Pyeloplasty, Nephrectomy, Cystectomy, Nephrostomy, Ureterostomy, Cystostomy (Suprapubic cystostomy), Urostomy, Nephrotomy, Nephropexy, Urethropexy, Lithotripsy, Kidney transplantation, and Renal biopsy. Non-limiting examples of plastic surgery include Abdominoplasty, Blepharoplasty, Phalloplasty, Mammoplasty, Buttock augmentation, Cryolipolysis, Cryoneuromodulation, Labiaplasty, Lip enhancement, Rhinoplasty, Otoplasty, Rhytidectomy, Genioplasty, Cheek augmentation, Orthognathic Surgery, Fillers injections, Brachioplasty, Liposuction, Zygoma reduction plasty, and Jaw reduction.

The methods and compositions of the present disclosure can also be used in a medical procedure in which an α2 adrenergic receptor agonist is used (e.g., colonoscopy, sigmoidoscopy, esophagogastroduodenoscopy, etc.)

The term “intraoperative”, as used herein, refers to any time period during the surgical procedure and prior to the end of the surgical procedure. In some embodiments, the end of the surgical procedure is when all manual and instrumental techniques being conducted on the subject for the surgical procedure have been completed. In other embodiments, the end of the surgical procedure is when the subject is removed from the operating room. The term “post-operative”, as used herein, refers to any time period after the end of the surgical procedure. In some embodiments, the term “post-operative” refers to any time following the surgical procedure when the subject is in the ICU.

The present disclosure provides methods to administer to a subject under general anesthesia or sedation an effective amount of an α2 adrenergic receptor antagonist in order to conduct an intraoperative assessment or post-operative assessment (e.g., clinical or physical examination). In one embodiment, the surgery is a neurosurgery, more specifically a craniotomy and the assessment performed is a standard motor and language mapping of the brain. Standard motor and language mapping of the brain are examples of cortical stimulation mapping, which is a highly invasive surgical procedure that involves the placement of electrodes on exposed brain tissue and delivery of an electric stimulus to identify the role of that part of the brain or to test the function of the part of the brain. In the context of a craniotomy, an assessment can be a standard motor mapping of the brain, wherein the standard motor mapping of the brain is the use of cortical stimulation mapping to stimulate a motor or sensory response within the body. In the context of a craniotomy, an assessment can be a standard language mapping, wherein the standard language mapping is the use of cortical stimulation mapping while a language-related assessment is performed on the subject. Non-limiting examples of language-related assessments are known to one of ordinary skill in the art and include reading sentences, completing sentences, auditory comprehension, answering basic questions, spontaneous speech, verb generation, fluency tasks (e.g., listing words that start with a specific letter), naming objects, listing letters, and counting. In some embodiment the standard language mapping is performed with functional magnetic resonance imaging (fMRI). In some aspects, the intraoperative assessment is an evaluation of the surgical procedure. For example the motor and language mapping of the brain when performed intraoperatively can reveal whether the preceding portions of the surgical procedure have resulted in any functional damage to the nervous system.

In the context of an orthopedic surgery, non-limiting examples of assessments are asking the subject to move a body part, asking a subject whether a body part can be felt, and asking a subject whether a physical stimulus can be felt. In the example of a spine surgery, the intraoperative assessment is an evaluation of the surgical procedure, because it reveals and functional damage sustained by the spine and any unintended pressure on the subject's spinal cord or nerves.

In some embodiments, an assessment is a measurement of the following parameters in a subject: heart rate, respiratory rate, blood pressure, urine output, knee reflex test, post-operative pain, and responsiveness to verbal communication.

In some embodiments a subject is administered α2 adrenergic receptor agonist and is intubated. As used herein, the term “intubated” or “intubation” refers to the insertion of a tube (e.g., endotracheal tube) into the trachea in order to connect a subject to a ventilator, which assists with breathing. Intubation is often used to assist with breathing when a subject is under general anesthesia or receiving certain types of sedation in critical care settings (e.g. ICU). As used herein, the term “extubate” or “extubation” refers to the removal of the endotracheal tube, often at the end of the surgery to allow the subject to breath unassisted. In some embodiments, the subject is administered α2 adrenergic receptor antagonist prior to the end of surgery. “Prior to the end of the surgical procedure”, as used herein, can be 0, 1, 2, 3, 4, 5, 6, 7, 8, 9, 10, 11, 12, 13, 14, 15, 16, 17, 18, 19, or 20 minutes prior to the end of surgery or in any range of time between 0 to 20 minutes prior to the end of surgery (e.g. 0-10 minutes, 0-15 minutes, etc.). The subject is then observed for a pharyngeal reflex (i.e. gag-reflex) before extubation.

Typically, when a subject does not have a gag-reflex towards the end or after the surgery, the clinicians conduct a trial extubation or a spontaneous breathing trial. In a trial extubation, the tube is removed before the subject displays a gag-reflex and is spontaneously breathing. It is done in the hope that the subject will begin breathing spontaneously. A failed trial extubation, may require reintubation. In a spontaneous breathing trial, the subject is still intubated and connected to the mechanical ventilator. The mechanical ventilator is set to minimal ventilator settings and the ability of the subject to begin their own breathing at these settings is observed. For such protocols, the patient is extubated if they pass the spontaneous breathing trial (i.e. demonstrate sufficient physiological evidence that they will be able to breath on their own without mechanical support). If not, the spontaneous breathing trial is repeated and/or lengthened. As explained above, the use of atipamezole would reduce or obviate the need for trial extubation and minimize time required for spontaneous breathing trials. It has the potential to reduce the number of failed extubations, and the potentially dangerous and expensive complications arising therefrom.

In some embodiments, a subject is administered an effective amount of an α2 adrenergic receptor agonist to perform a surgical procedure, then the subject is administered an effective amount of α2 adrenergic receptor antagonist prior to the end of the surgical procedure (or at the end of the surgical procedure), and the clinician observes the subject (performs an intraoperative assessment or post-operative assessment) for a brainstem reflex. Examples of brainstem reflexes include, but are not limited to, pharyngeal reflex, tracheal reflex, pupillary reflex, corneal reflex, spontaneous eye position and movements, occulocephalic (Doll's eye) reflex, occulovestibular (caloric) reflex, and jaw reflex.

In some embodiments, the post-operative assessment is conducted in the operating room. Alternatively, it is conducted after the patient has been transferred to the ICU. The post-operative assessments occurring after the end of the surgical procedure can occur within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 60 minutes of the end of surgery. In some embodiments, the postoperative assessment is conducted 1.5, 2, 2.5, or 3 hours after the end of the surgery.

### Cardiac Arrest

Patients receiving α2 adrenergic agonists can suffer from cardiovascular side-effects including reduced heart rate. It has been previously reported that patients can suffer from cardiac arrest when receiving dexmedetomidine. Currently, patients receive a standard regime of pharmacological agents to restart the heart including atropine and epinephrine. Contemplated in this disclosure is a α2-agonists adrenergic antagonist and methods for using the α2-agonists adrenergic antagonist to reduce the agent causing the cardiac arrest, which would identify the cause of the stopped heart as arising from the α2 adrenergic receptor agonist and restart the heart beating in these cases. As disclosed herein, the α2-agonists adrenergic antagonist can be used to determine if the cause of the cardiac arrest is due to the α2-receptor agonist drugs, which would be confirmed if the heart resumes beating. Thus, the methods used according to this disclosure allow the use of an α2 adrenergic receptor antagonist to diagnose/identify cardiac arrest intraoperatively. Additionally, the methods according to this disclosure allow the use of an α2 adrenergic receptor antagonist to treat cardiac arrest.

As used herein, the term “cardiac arrest” refers to the abrupt loss of heart function—whereby the heart ceases beating. In some cases it is preceded by abnormal heart rhythms, for example, an irregular heart rhythm, bradycardia (i.e., a depressed heart rate), tachycardia (i.e., an accelerated heart rhythm), and the like.

The term “treat,” as used herein refers to the process of administering an agent (e.g., therapeutic agent), wherein the object is to lessen or reverse an undesired physiological condition, disorder or disease, or to obtain beneficial or desired clinical results. The term “treat” implies that the agent elicits a clinically significant response, as determined by one of ordinary skill in the art.

In some aspects of the disclosure, a subject is in an inpatient setting or in an operating room receives α2 adrenergic agonist, alone or in combination with other sedative drugs. Upon the code alerting the clinical care team that the subject's heart has ceased beating, an α2-receptor antagonist is administered. In some embodiment, the α2 adrenergic receptor agonist is injected, infused, or oral. In some embodiments, the α2 adrenergic antagonist is administered as an injection. In some embodiments, the α2 adrenergic antagonist is administered as an infusion. In some embodiments, the α2 adrenergic antagonist is administered as either a single bolus, multiple boluses, or as a continuous infusion. In some embodiments, when the patient is under cardiac arrest, boluses of α2-adrenergic receptor antagonist are progressively administered until the patient's heart resumes beating.

In alternative embodiments, the methods of the present disclosure are used for treating bradycardia. As used herein, the term “bradycardia” refers to low heart rate or abnormally slow heart rate. The α2 adrenergic antagonist is administered to a subject under general anesthesia and experiencing bradycardia. Without being bound by theory, the administration of the α2 adrenergic antagonist reverses the effects of the agent causing the bradycardia.

In alternative embodiments, the methods of the present disclosure are used for treating hypotension. As used herein, the term “hypotension” refers to low blood pressure. The α2 adrenergic antagonist is administered to a subject under general anesthesia and experiencing hypotension. Without being bound by theory, the administration of the α2 adrenergic antagonist reverses the effects of the agent causing the hypotension.

### Circadian Rhythms

Circadian rhythms are the mental, physical, and behavioral changes that follow a 24-hour cycle and are driven by a master clock in the hypothalamus and environmental cues, such as daylight. Circadian rhythms are guided by day-light cycles. Chronic disruptions to a subject's circadian rhythms are correlated with secondary health problems, such as sleep disorders, obesity, diabetes, depression, and bipolar disorder.

In the ICU and other inpatient facilities, patients often suffer disruptions to their circadian rhythms, lose track of time and place, and as a result, can suffer from devastating delirium. It has been shown that delirium is linked to poor sleep and disruption of circadian rhythms. Sedation in the ICU for delirium typically relies on antipsychotic drugs such as haldol and quetiapine. More recently, α2 agonists such as dexmedetomidine (IV) or clonidine (oral) have shown promise as improved sedative agents for patients at risk for or suffering from delirium.

In one aspect of the present disclosure, a specific reversal agent for dexmedetomidine is administered to a subject, which enables proper initiating of circadian rhythms with a day-light cycle and reduces the risks of delirium onset and its persistence.

In some embodiments, a subject with delirium in the ICU would receive an α2 adrenergic receptor agonist for sleep at night time or as desired. Subsequently, to restart the circadian rhythms cycle and decrease the chance for delirium recurrence, the subject receives an α2 adrenergic antagonist agent to cause waking-up from their pharmacologically-aided sleep and properly begin their day-light schedule for initializing their circadian rhythms. In some embodiments, the α2 adrenergic receptor agonist is dexmedetomidine or clonidine. In some embodiments, the dexmedetomidine is administered as an injection. In some embodiments, the dexmedetomidine is administered intravenously. In some embodiments the clonidine is administered orally or by nasogastric tube. In some embodiments, the α2 adrenergic antagonist is atipamezole. In some embodiments, the α2-agonists adrenergic antagonist is administered as an injection. In some embodiment, the α2-agonists adrenergic antagonist is administered intravenously.

### Emergence Delirium

Upon emergence from general anesthesia, patients often become disoriented, combative, and disruptive, endangering themselves and, in extreme cases, the surrounding medical staff. This state is referred to as emergence delirium and can persist well after the patient leaves the operating room to another unit (e.g., ICU). The estimated incidence of emergence delirium, when a patient is waking up from general anesthesia, is 4-31% overall. In children, the incidence of emergence delirium has been estimated to be as high as 50-80%. As a result, many patients have to be restrained upon emergence from general anesthesia in the operating room and post-op unit (ICU, PACU, etc.).

In some embodiments, an α2 adrenergic antagonist is administered to prevent or reduce emergence delirium (i.e., the irritable, combative, inconsolable, or uncompromising, disorientation, emotional distress, and the physical manifestations thereof). In some embodiments, the α2 adrenergic receptor antagonist is administered before emergence (e.g., prophylactically). In some embodiments, the α2 adrenergic receptor antagonist is administered as the subject is regaining consciousness. In some embodiments, the α2 adrenergic receptor antagonist is administered as soon as the subject starts showing any symptoms of emergence delirium (e.g., distress, combativeness, etc.). In some embodiments, the α2 adrenergic receptor antagonist is administered by infusion. In alternative embodiments, the α2 adrenergic receptor antagonist is administered as a single bolus or multiple boluses (optionally rapidly administered boluses). These methods help reorient the patient, possibly through an increased state of arousal, to become less combative and minimize trauma during emergence from general anesthesia.

### Compositions and Administration

In some embodiments, α2 adrenergic receptor antagonist can be formulated in compositions for administration. In some embodiments, the α2 adrenergic receptor antagonist is a solution, for example, a saline/water-based solution.

The compounds described herein can be administered in combination with other therapeutic agents and such administration may be simultaneous or sequential. When the other therapeutic agents are administered simultaneously they can be administered in the same or separate formulations, but are administered at the same time. The administration of the other therapeutic agent can also be temporally separated, meaning that the therapeutic agents are administered at a different time, either before or after, the administration of the therapeutics described herein. The separation in time between the administration of these compounds may be a matter of minutes or it may be longer.

The invention described herein, in some aspects, includes methods for administering an α2 adrenergic receptor antagonist as a composition in the doses described herein, wherein the composition is a pharmaceutically acceptable composition. In embodiments where the composition is in a liquid form, a carrier can be a solvent or dispersion medium comprising but not limited to, water, and saline.

A “pharmaceutical composition” or “pharmaceutically acceptable composition” comprises the compound of the invention dissolved or dispersed in a pharmaceutically acceptable carrier. The term “pharmaceutical” or “pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans or mammals. This term requires that a compound or composition be nontoxic and sufficiently pure so that no further manipulation of the compound or composition is needed prior to administration to the subject.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers (e.g., antioxidants), gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection. In any case, the composition may comprise various antioxidants to retard oxidation of one or more components. Exemplary pharmaceutically acceptable carriers for α-adrenergic receptor antagonists in particular are described in U.S. Pat. No. 6,294,517.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The term “excipient”, as used herein, includes preservatives, suspending agents, stabilizers, dyes, buffers, antibacterial agents, antifungal agents, and isotonic agents, for example, sugars or sodium chloride. As used herein, the term “stabilizer” refers to a compound optionally used in the pharmaceutical compositions of the present invention in order to avoid the need for sulphite salts and increase storage life. Non-limiting examples of buffers include acetic acid, citric acid, boric acid, and phosphoric acid.

Preservatives can include anti-microbials, antioxidants, and agents that enhance sterility. Exemplary preservatives include ascorbic acid, ascorbyl palmitate, benzyl alcohol, BHA, BHT, citric acid, erythorbic acid, fumaric acid, malic 5 acid, propyl gallate, sodium ascorbate, sodium benzoate, sodium bisulfate, sodium metabisulfite, sodium sulfite, parabens (methyl-, ethyl-, butyl-), benzoic acid, potassium sorbate, and vanillin. The prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The composition of the present invention can comprise pharmaceutically acceptable salts. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, gluconic, and benzene sulphonic.

The compositions may take such forms as suspensions, solutions or emulsions in oily or aqueous vehicles, and may contain formulatory agents such as suspending, stabilizing and/or dispersing agents.

In some embodiments, the α2-adrenergic receptor antagonist is administered as a bolus or as an infusion. Bolus, as used herein, is a defined, discrete amount of a substance (e.g., a dose) delivered intravenously, intramuscularly, intrathecally, or subcutaneously. A bolus can also be a dose delivered by inhalation. In some embodiments, a subject is given one bolus of the α2-adrenergic receptor antagonist. In further embodiments, the subject is given more than one bolus of the α2-adrenergic receptor antagonist, wherein the more than one bolus of the α2-adrenergic receptor antagonist is 2, 3, 4, or 5 boluses of an α2 adrenergic receptor antagonist, in order to facilitate emergence from sedation or unconsciousness. The boluses can be administered during a surgical procedure, immediately after a surgical procedure, over the course of a day, or over the course of multiple days. In some embodiments, the subject is given more than 5 boluses of the α2-adrenergic receptor antagonist over the course of multiple days. In the case of cardiac arrest, where urgency is needed, the α2-adrenergic receptor antagonist needs to be administered rapidly and/or in the form of multiple boluses. Alternatively or in addition, the medication to reverse unconsciousness or sedation is administered by infusion, such as intravenous infusion. Preferably the material is infused into the body (e.g., via intravenous means), but could also be administered by other delivery methods. For instance, the compounds of the present invention can be administered by any available or effective delivery method including, but not limited to, intravenously, intradermally, intraarterially, intralesionally, intratumorally, intracranially, intraarticularly, intraprostaticaly, intrapleurally, intratracheally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, transdermal drug delivery, injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). In some embodiments, the α2-adrenergic receptor antagonist is administered as a buccal spray. In preferred embodiments, the methods and compositions of the present disclosure involve intravenous administration of the α2-adrenergic receptor antagonist.

The compounds of the invention may be administered directly to a tissue. Direct tissue administration may be achieved by direct injection. In some embodiment, the compounds of the invention are administered intravenously. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

### Dosing

The invention described herein, in some aspects, includes methods for administering to a subject that is under general anesthesia or sedation, an effective amount of an α2 adrenergic receptor antagonist. The term “therapeutically effective amount” or “effective amount” as used herein, refers to the amount of active compound or pharmaceutical agent that elicits a biological or medicinal response in a tissue, system, animal, individual or human that is being sought by a researcher, veterinarian, medical doctor or other clinician, which includes one or more of the following: (1) the amount sufficient for the subject to regain full consciousness, (2) the amount sufficient to regain any motor and/or cognitive faculties required to perform the intraoperative or post-operative assessment on the subject, (3) the amount sufficient to make a subject ambulatory while staying in an ICU, (4) the amount sufficient to reverse cardiac arrest occurring during a surgical procedure. The effective amount of a compound of the invention described herein (e.g., an α2 adrenergic receptor antagonist) may vary depending upon the specific compound used, the mode of delivery of the compound, and whether it is used alone or in combination. The effective amount for any particular application can also vary depending on such factors as the disease being assessed or treated, the particular compound being administered, the size of the subject, the severity of the disease or condition, the detection method, as well as the amount of anesthetic/sedation agent used. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective regimen can be planned.

Multiple doses of α2 adrenergic receptor antagonist are contemplated herein. In some embodiments, the dose range of α2 adrenergic receptor antagonist given subsequent to the administration of the anesthetic or sedative is 30:1-400:1: μg/kg (antagonist:agonist). In some embodiments, the dose range is 5:1, 10:1, 15:1, 20:1, 25:1, 30:1, 35:1, 37:1, 38:1, or 39:1 μg/kg. In some embodiments, the dose of α2 adrenergic receptor antagonist given subsequent to the administration of the anesthetic or sedative is 40:1, 41:1, 42:1, 43:1, 44:1, 45:1, 46:1, 47:1, 48:1, 49:1, 50:1, 51:1, 52:1, 53:1, 54:1, 55:1: 56:1, 57:1, 58:1, 59:1, 60:1, 62:1, 64:1, 66:1, 68:1, 70:1, 72:1, 74:1, 76:1, 78:1, 80:1, 85:1, 90:1, 95:1, 100:1, 110:1, 120:1, 130:1, 140 150:1, 160:1, 170:1, 180:1, 190:1, 200:1, 220:1, 240:1, 260:1, 280:1, 300:1, 320:1, 340:1, 360:1, 380:1, 400:1, 420:1, 440:1, 460:1, 480:1, or 500:1 μg/kg. In some embodiments, the dose range is 5:1-20:1, 5:1-30:1, 5:1-50:1, 5:1-70:1, 5:1-100:1, 5:1-100:1, 20:1-40:1, 20:1-60:1, 20:1-80:1, 20:1-100:1, 20:1-150:1, 20:1-200:1, 20:1-400:1, 30:1-60:1, 30:1-80:1, 30:1-100-1, 30:1-125:1, 30:1-150:1, 30:1-175:1, 30:1-200:1, 30:1-250:1, 30:1-300:1, 30:1-350:1, 30:1-400:1, 30:1-450:1, 30:1-500:1, 40:1-60:1, 40:1-80:1, 40:1-100-1, 40:1-125:1, 40:1-150:1, 40:1-175:1, 40:1-200:1, 40:1-250:1, 40:1-300:1, 40:1-350:1, 40:1-400:1, 40:1-500:1, 80:1-100:1, 80:1-120:1, 80:1-140:1, 80:1-160:1, 80:1-180:1, 80:1-200:1, 80:1-225:1, 80:1-250:1, 80:1-275:1, 80:1-300:1, 80:1-350:1, 80:1-400:1, 80:1-500:1, 100:1-120:1, 100:1-140:1, 100:1-160:1, 100:1-180:1, 100:1-200:1, 100:1-240:1, 100:1-280:1, 100:1-300:1, 100:1-325:1, 100:1-350:1, 100:1-375:1, 100:1-400:1, 100:1-500:1, 150:1-175:1, 150:1-200:1, 150:1-225:1, 150:1-250:1, 150:1-275:1, 150:1-300:1, 150:1-325:1, 150:1-350:1, 150:1-375:1, 150:1-400:1, 150:1-425:1, 150:1-450:1, 150:1-500:1, 200:1-250:1, 200:1-300:1, 200:1-350:1, 200:1-400:1, 200:1-450:1 or 200:1-500:1 μg/kg (antagonist:agonist). In some embodiments, the α2 adrenergic receptor antagonist is atipamezole.

In some embodiments, the pre-anesthetic dose is an α2 adrenergic receptor agonist (e.g. dexmedetomidine) and can range from 0.15 μg/kg to 2 μg/kg. In some embodiments 0.05, 0.10, 0.15, 0.20, 0.25, 0.30, 0.30, 0.35, 0.40, 0.45, 0.50, 0.55, 0.60, 0.65, 0.70, 0.75, 0.80, 0.85, 0.90, 0.95, 1.00, 1.25, 1.50, 1.75, 2.00, 2.25, 2.50, 2.75, 3.00, 3.5, 4.00, 4.50, or 5.00 μg/kg.

In some embodiments, the α2 adrenergic receptor antagonist is administered by infusion at rates ranging from 0.2-2 μg/kg/hr. In some embodiments, the α2 adrenergic receptor antagonist is administered by infusion at rates up to 20 μg/kg/min. In some embodiments, the α2 adrenergic receptor antagonist is administered by infusion at 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 6, 7, 8, 9, 10, 11, 12, 13, 14, 15, 16, 17, 18, 19, or 20 μg/kg/min. In some embodiments, the α2 adrenergic receptor antagonist is administered by infusion at a rate greater than 20 μg/kg/min. In alternative embodiments, the α2 adrenergic receptor antagonist is administered by bolus.

Generally, the dosage required to constitute an effective amount can be adjusted by one of ordinary skill in the art. The dosage can also vary from one subject to another, based on age, health, physical condition, sex, weight, and contraindications.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### OTHER EMBODIMENTS

### Embodiment 1

A method comprising:

a) administering to a subject an effective amount of an α2 adrenergic receptor agonist to perform a surgical procedure;

b) inducing a planned wake-up by administering to the subject an effective amount of α2 adrenergic receptor antagonist, wherein the adrenergic receptor agonist is maintained or reduced;

c) conducting an intraoperative assessment of the subject during the planned wakeup; and

d) ceasing the administration of the α2 adrenergic receptor antagonist in order to proceed with the surgical procedure.

### Embodiment 2

The method of embodiment 1, wherein the intraoperative assessment is performed in an operating room.

### Embodiment 3

The method of embodiment 1, wherein after ceasing the α2 adrenergic receptor antagonist, the amount of the adrenergic receptor agonist is increased to its amount at the initiation of the surgical procedure.

### Embodiment 4

The method of embodiment 1 or embodiment 3, wherein the surgical procedure is a neurosurgery or an orthopedic surgery.

### Embodiment 5

The method of embodiment 4, wherein the neurosurgery is a craniotomy.

### Embodiment 6

The method of embodiment 4, wherein the orthopedic surgery is a spine surgery.

### Embodiment 7

The method of any one of embodiments 1-3, wherein the intraoperative assessment is a physiologic or functional assessment of an organ, organ system, or anatomical region.

### Embodiment 8

The method of any one of embodiments 1-3, wherein the intraoperative assessment is an evaluation of the surgical procedure.

### Embodiment 9

The method of any one of embodiments 1-3, wherein the intraoperative assessment is to assess the subject's breathing.

### Embodiment 10

The method of embodiment 4 or embodiment 5, wherein the intraoperative assessment is a standard motor and language mapping of the subject's brain.

### Embodiment 11

The method of embodiment 4 or embodiment 6, wherein the intraoperative assessment is a standard motor mapping technique of the subject's spinal cord or musculature.

### Embodiment 12

The method of embodiment 1, further comprising administering to the subject an effective amount of a general anesthetic.

### Embodiment 13

A method comprising:

a) administering to a subject undergoing a surgical procedure an effective amount of an α2 adrenergic receptor agonist to perform a surgical procedure,

b) ceasing the administration of the α2 adrenergic receptor agonist and administering to the subject an effective amount of α2 adrenergic receptor antagonist at or near the end of the surgery, and

c) conducting a post-operative assessment.

### Embodiment 14

The method of embodiment 13, wherein the post-operative assessment is conducted within 30 minutes of the end of the surgery.

### Embodiment 15

The method of embodiment 13, wherein the post-operative assessment is conducted in the operating room after the end of surgery.

### Embodiment 16

The method of embodiment 13 or embodiment 14B, wherein the post-operative assessment is conducted within 10 minutes of the end of surgery.

### Embodiment 17

The method of 13, wherein the post-operative assessment is conducted within 1 hour of the end of surgery.

### Embodiment 18

A method comprising administering to a subject undergoing a surgical procedure an effective amount of an α2 adrenergic receptor agonist to perform a surgical procedure, ceasing the administration of the α2 adrenergic receptor agonist and administering to the subject an effective amount of α2 adrenergic receptor antagonist at or near the end of the surgery, wherein the subject's mobility in an intensive or critical care unit is increased.

### Embodiment 19

The method of embodiment 18, wherein the subject's mobility is increased within 90 minutes of the end of the surgery.

### Embodiment 20

A method of modifying circadian rhythms, the method comprising administering to a subject in need thereof, treated with an α2 adrenergic agonist, an effective amount of an α2 adrenergic receptor antagonist in order to facilitate normal adaptation to regular day-light cycles or adjust the subject's circadian rhythms.

### Embodiment 21

The method of embodiment 20, wherein the subject is a patient at risk of delirium.

### Embodiment 22

The method of embodiment 20 or embodiment 21, wherein the α2 adrenergic receptor agonist is administered for sleep at night time or as desired.

### Embodiment 23

The method of any one of embodiments 20-22, wherein the α2 adrenergic receptor antagonist is administered to restart a circadian rhythms cycle.

### Embodiment 24

A method of treating cardiac arrest, the method comprising administering to a subject under general anesthesia and experiencing cardiac arrest, an effective amount of an α2 adrenergic receptor antagonist to treat the cardiac arrest, wherein the subject under general anesthesia had received an α2 adrenergic receptor agonist.

### Embodiment 25

A method of treating bradycardia, the method comprising administering to a subject under general anesthesia and experiencing bradycardia, an effective amount of an α2 adrenergic receptor antagonist to treat the bradycardia, wherein the subject under general anesthesia had received an α2 adrenergic receptor agonist.

### Embodiment 26

A method of treating hypotension, the method comprising administering to a subject under general anesthesia and experiencing hypotension, an effective amount of an α2 adrenergic receptor antagonist to treat the hypotension, wherein the subject under general anesthesia had received an α2 adrenergic receptor agonist.

### Embodiment 27

A method comprising:

a) administering to a subject an effective amount of an α2 adrenergic receptor agonist to perform a surgical procedure, wherein the subject is given an endotracheal breathing tube;

b) ceasing the administration of the α2 adrenergic agonist;

(c) administering to the subject an effective amount of α2 adrenergic receptor antagonist prior to the end of the surgical procedure; and

(d) conducting an extubation when the subject displays a brainstem reflex.

### Embodiment 28

The method of embodiment 27, wherein (c) is conducted between 0 and 15 minutes before the end of the surgical procedure.

### Embodiment 29

The method of embodiment 27, wherein the brainstem reflex is a pharyngeal reflex.

### Embodiment 30

The method of embodiment 27, wherein the brainstem reflex is a tracheal reflex.

### Embodiment 31

A method comprising:

a) administering to a subject an effective amount of an α2 adrenergic receptor agonist to perform a surgical procedure;

b) ceasing the administration of the α2 adrenergic agonist;

(c) administering to the subject an effective amount of α2 adrenergic receptor antagonist prior to the end of the surgical procedure; and

(d) conducting an intraoperative or post-operative assessment to determine if the subject displays a brainstem reflex.

### Embodiment 32

The method of embodiment 31, wherein (c) is conducted between 0 and 15 minutes before the end of the surgical procedure.

### Embodiment 33

The method of embodiment 31, wherein (c) is conducted after the end of the surgical procedure.

### Embodiment 34

The method of embodiment 33, wherein (c) is conducted within 30 minutes after the end of the surgical procedure.

### Embodiment 35

A method for reversing a neural blockade during a surgical procedure, comprising:

administering to a subject undergoing a surgical procedure who has received a muscle blockade agent and a neural blockade agent, an effective amount of an α2 adrenergic receptor antagonist to reverse the neural blockade.

### Embodiment 36

The method of embodiment 35, further comprising conducting an intraoperative assessment of the subject following the administration of the α2 adrenergic receptor antagonist.

### Embodiment 37

The method of embodiment 35 or 36, wherein the α2 adrenergic receptor antagonist is given in a dose range of 30:1-400:1 μg/kg α2 adrenergic receptor antagonist to neural blockade agent.

### Embodiment 38

A method for reducing or preventing emergence delirium, the method comprising:

administering to a subject under general anesthesia, an effective amount of an α2 adrenergic receptor antagonist, wherein the subject under general anesthesia had received an α2 adrenergic receptor agonist.

### Embodiment 39

The method of embodiment 38, wherein the α2 adrenergic receptor antagonist is administered before emergence from general anesthesia.

### Embodiment 40

The method of embodiment 39, wherein the α2 adrenergic receptor antagonist is administered as the subject begins to regain consciousness.

### Embodiment 41

The method of embodiment 39, wherein the α2 adrenergic receptor antagonist is administered in an effective amount to stop at least one symptom of emergence delirium.

### Embodiment 42

The method of any one of embodiment 1-40, wherein the α2 adrenergic receptor antagonist is given in a dose range of 30:1-400:1 μg/kg α2 adrenergic receptor antagonist to α2 adrenergic agonist.

43. The method of any one of embodiments 1-42, wherein α2 adrenergic receptor antagonist provides rapid reversal of unconsciousness or sedation.

44. The method of any one of embodiments 1-43, wherein the α2 adrenergic receptor antagonist is atipamezole.

45. The method of any one of embodiments 1-44, wherein the α2 adrenergic receptor antagonist is administered intravenously.

46. The method of any one of embodiments 1-45, wherein the α2 adrenergic receptor antagonist is a composition comprising the α2 adrenergic receptor antagonist and a pharmaceutically acceptable excipient.

### Embodiment 47

The method of embodiment 46, wherein the pharmaceutically acceptable excipient is a saline or water-based solution.

### Embodiment 48

The method of any one of embodiments 1-47, wherein the α2 adrenergic receptor antagonist is administered intravenously as a bolus or an infusion.

### Embodiment 49

The method of any one of embodiments 1-38, wherein the α2 adrenergic receptor agonist is dexmedetomidine or clonidine.

### Embodiment 50

The method of embodiment 49, wherein the α2 adrenergic receptor agonist is administered in solo or in combination with other general anesthetics or sedative drugs.

### Embodiment 51

The method of embodiment 38, wherein the general anesthetic is propofol, sevoflurane, ketamine, or benzodiazepines.

### Embodiment 52

The method of any one of embodiments 1-51, wherein the subject is a mammal.

### Embodiment 53

The method of any one of embodiments 1-52, wherein the subject is a human.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

1. A method comprising:
a) administering to a subject an effective amount of an α2 adrenergic receptor agonist to perform a surgical procedure;
b) inducing a planned wake-up by administering to the subject an effective amount of α2 adrenergic receptor antagonist, wherein the adrenergic receptor agonist is maintained or reduced;
c) conducting an intraoperative assessment of the subject during the planned wakeup; and
d) ceasing the administration of the α2 adrenergic receptor antagonist in order to proceed with the surgical procedure.

2. The method of claim 1, wherein the intraoperative assessment is performed in an operating room.

3. The method of claim 1, wherein after ceasing the α2 adrenergic receptor antagonist, the amount of the adrenergic receptor agonist is increased to its amount at the initiation of the surgical procedure.

4. The method of claim 1, wherein the surgical procedure is a neurosurgery such as a craniotomy or an orthopedic surgery such as a spine surgery.

5. The method of claim 1, wherein the intraoperative assessment is a physiologic or functional assessment of an organ, organ system, or anatomical region, an evaluation of the surgical procedure, to assess the subject's breathing, a standard motor and language mapping of the subject's brain, or a standard motor mapping technique of the subject's spinal cord or musculature.

6. The method of claim 1, further comprising administering to the subject an effective amount of a general anesthetic.

7. A method comprising:
a) administering to a subject undergoing a surgical procedure an effective amount of an α2 adrenergic receptor agonist to perform a surgical procedure,
b) ceasing the administration of the α2 adrenergic receptor agonist and administering to the subject an effective amount of α2 adrenergic receptor antagonist at or near the end of the surgery, and
c) conducting a post-operative assessment.

8. The method of claim 7, wherein the post-operative assessment is conducted within 30 minutes of the end of the surgery, in the operating room after the end of surgery, within 10 minutes of the end of surgery, or within 1 hour of the end of surgery.

9. The method of claim 7, wherein the subject's mobility in an intensive or critical care unit is increased, optionally wherein the subject's mobility is increased within 90 minutes of the end of the surgery.

10. A method of modifying circadian rhythms, the method comprising administering to a subject in need thereof, treated with an α2 adrenergic agonist, an effective amount of an α2 adrenergic receptor antagonist in order to facilitate normal adaptation to regular day-light cycles or adjust the subject's circadian rhythms.

11. The method of claim 10, wherein the subject is a patient at risk of delirium.

12. The method of claim 10, wherein the α2 adrenergic receptor agonist is administered for sleep at night time or as desired.

13. The method of claim 10, wherein the α2 adrenergic receptor antagonist is administered to restart a circadian rhythms cycle.

14-16. (canceled)

17. The method of claim 7,
wherein the subject is given an endotracheal breathing tube; administering to the subject an effective amount of α2 adrenergic receptor antagonist prior to the end of the surgical procedure and optionally between 0 and 15 minutes before the end of the surgical procedure; and (d) conducting an extubation when the subject displays a brainstem reflex, optionally wherein the brainstem reflex is a pharyngeal reflex or a tracheal reflex.

18. The method of claim 7, further comprising
administering to the subject an effective amount of α2 adrenergic receptor antagonist prior to the end of the surgical procedure; and conducting an intraoperative or post-operative assessment to determine if the subject displays a brainstem reflex.

19. (canceled)

20. A method for reversing a neural blockade during a surgical procedure, comprising:
administering to a subject undergoing a surgical procedure who has received a muscle blockade agent and a neural blockade agent, an effective amount of an α2 adrenergic receptor antagonist to reverse the neural blockade.

21. The method of claim 20, further comprising conducting an intraoperative assessment of the subject following the administration of the α2 adrenergic receptor antagonist.

22. The method of claim 20, wherein the α2 adrenergic receptor antagonist is given in a dose range of 30:1-400:1 μg/kg α2 adrenergic receptor antagonist to neural blockade agent.

23. A method for reducing or preventing emergence delirium, the method comprising:
administering to a subject under general anesthesia, an effective amount of an α2 adrenergic receptor antagonist, wherein the subject under general anesthesia had received an α2 adrenergic receptor agonist, optionally wherein the α2 adrenergic receptor antagonist is given in a dose range of 30:1-400:1 μg/kg α2 adrenergic receptor antagonist to α2 adrenergic agonist.

24. The method of claim 23, wherein the α2 adrenergic receptor antagonist is administered before emergence from general anesthesia, as the subject begins to regain consciousness, or in an effective amount to stop at least one symptom of emergence delirium.

25. The method of claim 1, wherein α2 adrenergic receptor antagonist provides rapid reversal of unconsciousness or sedation.

26. The method of claim 1, wherein the α2 adrenergic receptor antagonist is atipamezole.

27. The method of claim 1, wherein the α2 adrenergic receptor antagonist is administered intravenously.

28. The method of claim 1, wherein the α2 adrenergic receptor antagonist is a composition comprising the α2 adrenergic receptor antagonist and a pharmaceutically acceptable excipient.

29. The method of claim 1, wherein the α2 adrenergic receptor agonist is dexmedetomidine or clonidine.

30. The method of claim 29, wherein the α2 adrenergic receptor agonist is administered in solo or in combination with other general anesthetics or sedative drugs, such as propofol, sevoflurane, ketamine, or benzodiazepines.
